(19) United States
(12) Patent Application Publication
(10) Pub. No.: US 2026/0306830 A1
(43) Pub. Date: Oct. 8, 2026

(54) TRANSFORMABLE STRUCTURES FOR THERAPY

(71) Applicant: Medtronic, Inc., Minneapolis, MN (US)

(72) Inventor: Chandrasekhar R. Rajagopalan, Minneapolis, MN (US)

(21) Appl. No.: 19/479,617

(22) PCT Filed: Apr. 15, 2024
(86) PCT No.: PCT/IB2024/053672

§ 371 Date: Oct. 28, 2025

Related U.S. Application Data
(60) Provisional application No. 63/498,973, filed on Apr. 28, 2023.

(51) Int. Cl. A61N 1/05; A61N 1/40

(52) U.S. Cl. CPC A61N 1/0529; A61N 1/40

(57) ABSTRACT

Devices, systems, and techniques are disclosed for delivering electrical stimulation therapy and/or sensing of physiological signals via electrodes disposed on transformable auxetic structures. For example, a system may include a structure comprising a pattern of repeating unit cells configured to convert the structure between a compact configuration and an expanded configuration. The structure may be substantially flat in the compact configuration. The structure may define one or more three-dimensional contours in the expanded configuration. Each unit cell of the repeating unit cells may comprise a plurality of elements having increased distances between adjacent elements of the plurality of elements in the expanded configuration as compared to the compact configuration. One or more electrodes may be disposed on the structure.

This Application claims priority from U.S. Provisional Patent Application 63/498,973, filed 28 Apr. 2023, the entire content of which is incorporated herein by reference.

### TECHNICAL FIELD

The disclosure relates to implantable systems for delivering electrical stimulation and/or sensing electrical signals.

### BACKGROUND

Implantable medical devices may be configured to deliver electrical stimulation therapy and/or monitor physiological signals. Electrical stimulation of neural tissue, for example, may provide relief for a variety of disorders, improving the quality of life for many patients. Some implantable medical devices may employ elongated electrical leads that carry electrodes. For example, electrodes may be located at a distal portion of a lead. A proximal portion of the lead may include electrical contacts that are coupled to the electrodes and coupled to terminal in an implantable medical device housing, which may contain electronic circuitry such as electrical stimulation generation circuitry and/or sensing circuitry. In some examples, stimulation may be conducted using multiple electrodes and multiple stimulation leads in order to provide electrical stimulation at multiple positions within the patient or to provide electrical stimulation to a targeted area. One example of electrical stimulation therapy includes alternating electric field (AEF) therapy or focused tumor treating field (FTTF) therapy, a type of electric field therapy which uses low-intensity electrical fields to treat brain tumors.

### SUMMARY

In general, the disclosure describes devices, systems, and techniques related to delivery of electrical stimulation to target treatment areas within a patient using electrodes and/or leads carried by or disposed on a transformable auxetic structure. Auxetic structures and metamaterial architectures enable manufacturing of structures, including defining locations for electrode placement, in a two-dimensional or planar space, or simpler geometries (e.g., when the structure is substantially flat and in a compact configuration), and a deployment and/or expansion to define one or more three-dimensional contours (e.g., in an expanded configuration). The three-dimensional contours may be pre-determined contours and determined to correspond to a portion of the anatomy of a patient, such as from one or more imaging modalities (e.g., magnetic resonance imaging (MRI), computed tomography (CT), etc.).

In some examples, auxetic structures include a number of unit cells arranged in such a way (e.g., a pattern of repeating unit cells and/or a pattern of repeating auxetic unit cells) that the overall structure is configured to convert from a collapsed configuration to an expanded configuration when stretched (e.g., a tensile force or tension force). This unusual deformation may give rise to a negative Poisson's ratio and altered mechanical properties. In some examples, the unit cells include a plurality of elements within the unit cell that have increased distances between adjacent elements in the expanded configuration as compared to the compact configuration.

In some examples, electrodes disposed on the transformable auxetic structure may be configured to deliver electric field therapy, which may be used for various reasons, such as reducing or preventing the growth of tumor cells, such as glioblastomas, or the reduction in growth or proliferation of non-tumorous cells within the body. Electric field therapy may include modulated electrical field therapy which may include types of electrical field modulation, such as alternating electrical field (AEF) or focused tumor treating field (FTTF) therapy. The electrodes may additionally or alternatively be configured to sense electrical signals from the patient.

In an example, a system includes: a structure comprising a pattern of repeating unit cells configured to convert the structure between a compact configuration and an expanded configuration, wherein: the structure is substantially flat in the compact configuration, the structure defines one or more three-dimensional contours in the expanded configuration, and each unit cell of the repeating unit cells comprises a plurality of elements having increased distances between adjacent elements of the plurality of elements in the expanded configuration as compared to the compact configuration; and one or more electrodes disposed on the structure.

In another example, a system includes: a bi-stable auxetic structure comprising a pattern of repeating auxetic unit cells configured to convert the bi-stable auxetic structure between a compact configuration and an expanded configuration upon application of a tension force to the bi-stable auxetic structure, wherein: the bi-stable auxetic structure is substantially flat in the compact configuration, the bi-stable auxetic structure defines one or more three-dimensional contours in the expanded configuration, each auxetic unit cell of the pattern of repeating auxetic unit cells comprises a plurality of elements having increased distances between adjacent elements of the plurality of elements in the expanded configuration as compared to the compact configuration, the compact configuration is a first stable configuration, the expanded configuration is a second stable configuration, and the bi-stable structure is unstable in configurations between the first stable configuration and the second stable configuration; and one or more electrodes disposed on the structure in one or more locations corresponding to pre-determined target locations within a patient for therapy delivery.

In another example, a method includes: generating a structure, the structure comprising a pattern of repeating unit cells configured to convert the structure between a compact configuration and an expanded configuration, wherein: the structure is substantially flat in the compact configuration, the structure defines one or more three-dimensional contours in the expanded configuration, and each unit cell of the repeating unit cells comprises a plurality of elements having increased distances between adjacent elements of the plurality of elements in the expanded configuration as compared to the compact configuration; and attaching one or more electrodes to the structure.

In another example, a method includes: transforming a transformable structure to an expanded configuration; inserting the transformable structure into a patient; receiving, via processing circuitry, a request to deliver therapy; and delivering therapy via one or more electrodes disposed on the transformable structure, wherein: the structure comprises a pattern of repeating unit cells configured to convert the structure between a compact configuration and an expanded configuration, the structure is substantially flat in the compact configuration, the structure defines one or more three-dimensional contours in the expanded configuration, and each unit cell of the repeating unit cells comprises a plurality of elements having increased distances between adjacent elements of the plurality of elements in the expanded configuration as compared to the compact configuration; and one or more electrodes disposed on the structure.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an example transformable structure with one or more electrodes disposed on the structure.

FIG. 2 is a block diagram of the example IMD of FIG. 1 for delivering therapy according to an example of the techniques of the disclosure.

FIG. 3 is a block diagram of the external programmer of FIG. 1 for controlling delivery of therapy according to an example of the techniques of the disclosure.

FIG. 4 is a conceptual diagram illustrating example configurations of an example transformable structure.

FIG. 5 is a conceptual diagram illustrating an example IMD and a side section view of an example transformable structure.

FIG. 6 is a conceptual diagram illustrating an example IMD and a side section view of an example transformable structure.

FIG. 7 is a flow diagram illustrating an example technique to manufacture an example system.

FIG. 8 is a flow diagram illustrating an example technique for implanting and/or inserting a transformable structure into a patient.

Like reference characters denote like elements throughout the description and figures.

### DETAILED DESCRIPTION

In accordance with the techniques of this disclosure, a system may include a transformable structure that carries one or more electrodes or one or more leads, wherein the electrodes configured to delivery electrical stimulation therapy to a target location proximate the brain of a patient when the transformable structure is implanted in the patient.

Electrodes may be placed in tissues of a patient for delivering electrical signals to tissue. For example, a lead may be inserted into the tissue, where the lead includes several electrodes along the length of the lead. Such leads may be referred to as cylindrical leads carrying multiple ring electrodes. However, in some situations, the desired treatment or target tissues may benefit from electric fields provided at different locations within the tissue or around a cavity within tissue. Cylindrical leads may not be easily deployable and/or maintained to provide stimulation at these locations.

As described herein, auxetic structures and metamaterial architectures enable manufacturing of structures, including defining locations for electrode placement, in a two-dimensional or planar space (e.g., when the structure is substantially flat and in a compact configuration), and a deployment and/or expansion to define one or more three-dimensional contours (e.g., in an expanded configuration). The electrodes can then remain on or attached to respective portions of the auxetic structures and used for stimulation or sensing.

Auxetic structures can include a number of unit cells arranged in such a way (e.g., a pattern of repeating unit cells and/or a pattern of repeating auxetic unit cells) that the overall structure is configured to convert from a collapsed configuration to an expanded or deployed configuration in response to a directed force (e.g., a tensile force or a compressive force). In other words, these auxetic structures may be manufactured in a flat, or two-dimensional (2D) space and then transformed from the flat undeployed compact configuration to the three-dimensional (3D) deployed or expanded configuration. In some examples, the unit cells include a plurality of elements within the unit cell that have increased distances between adjacent elements in the expanded configuration as compared to the compact configuration. In some examples, unit cells of different sizes and building block geometries are incorporated in the compact configuration to achieve the desired 3D shape in the expanded configuration.

Although this disclosure is directed to structures implanted proximate the brain of a patient for delivery of AEF or FTTF therapy for the purpose of treating glioblastoma, the systems, devices, and techniques described herein may similarly operate to deliver AEF or FTTF therapy or similar electric-field therapies to other tissue areas and/or to treat different types of cancer. For example, a system may be implanted to treat and/or prevent cancer in the spine, pelvis, abdomen, or any other location. Some examples of target tissue may include regions of expected metastatic elements, such as lymph nodes, to reduce the spread of cells from a different tumor cite. Structures described herein may be used in applications in which therapy is delivered to areas of complex anatomical geometry. In other examples, the electrodes carried by a transformable structure may be configured to sense signals or deliver electrical stimulation for other purposes, such as treating pain or deep brain stimulation therapy to modulate neurological function in the brain, or in any other location of the body. Moreover, a human patient is described for example purposes herein, but similar systems, devices, and techniques may be used for other animals in other examples.

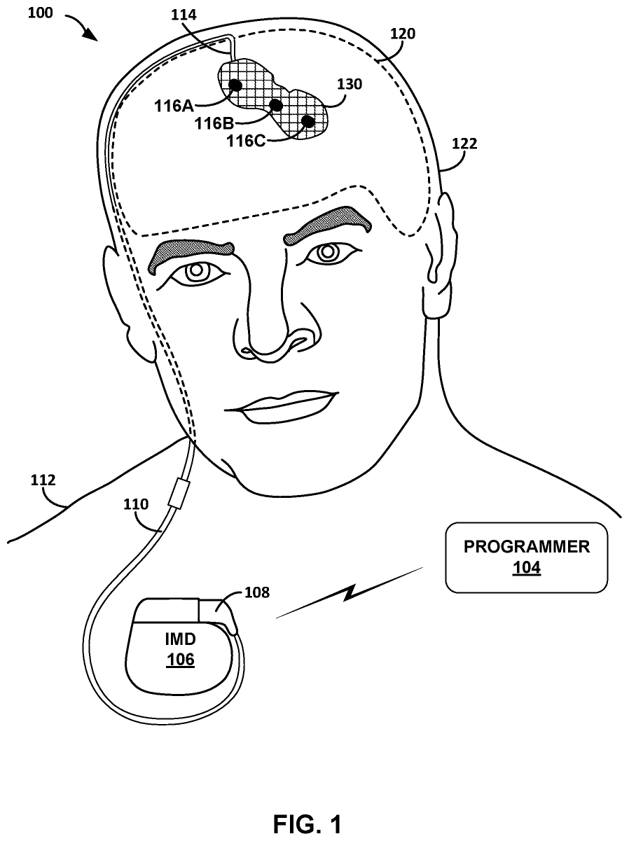

FIG. 1 is a conceptual diagram illustrating an example system 100 that includes an implantable medical device (IMD) 106 configured to deliver therapy to patient 112 according to an example of the techniques of the disclosure. This therapy may be AEF or FTTF therapy or another therapy based on applied electrical fields. As shown in the example of FIG. 1, example system 100 includes medical device programmer 104, implantable medical device (IMD) 106, lead extension 110, lead 114, and electrodes 116A, 116B, and 116C (hereinafter collectively referred to as “electrodes 116”), plurality of electrodes 116, electrode array 116, or one or more electrodes 116). Electrodes 116 and/or lead 114 may be disposed on, carried by, or otherwise affixed to by a structure 130, which may be a deployable auxetic structure. In the example shown in FIG. 1, electrodes 116 are positioned to deliver electrical stimulation to a tissue site within brain 120, such as a deep brain site under the dura mater of brain 120 of patient 112. In some examples, electric fields (e.g., electrical stimulation) are delivered to one or more regions of brain 120, such as a region that contains a tumor such as glioblastoma, or region from which a glioblastoma was resected (removed). In the example of a resection cavity where the tumor was removed, e.g., the cavity within a tumor bed, may be or be part of the target tissue for AEF or FTTF therapy. The tumor bed may be of various sizes, but may be between approximately 1 mm to 3 mm in diameter in some examples. Some or all of electrodes 116 also may be positioned to sense neurological brain signals within brain 120 of patient 112. In some examples, some of electrodes 116 may be configured to sense neurological brain signals, impedance, etc., and some or all of electrodes 116 may be configured to deliver electrical stimulation to brain 120 in the form of AEF or FTTF therapy. In other examples, all of electrodes 116 are configured to both sense electrical signals and deliver electrical stimulation to brain 120.

IMD 106 includes a therapy module (e.g., which may include processing circuitry, signal generation circuitry or other electrical circuitry configured to perform the functions attributed to IMD 106) that includes a stimulation generator configured to generate and deliver electrical stimulation therapy (e.g., AEF or FTTF therapy) to patient 112 via a subset (e.g., less than all) of electrodes 116. The subset of electrodes 116 that are used to deliver electrical stimulation to patient 112, and, in some cases, the polarity of the subset of electrodes 116 may be referred to as a stimulation electrode combination. As described in further detail below, the stimulation electrode combination can be selected for a particular patient 112 and target tissue site (e.g., selected based on the patient condition or based on the determined location of a tumor or other tissue of interest). The group of electrodes 116 includes at least one electrode and can include more than one electrode. Further, while the example of FIG. 1 includes one lead (e.g., lead 114), more than one lead may be used. Electrodes 116 may be a portion of structure 130, or electrodes can be coupled to structure 130. In some examples, electrodes 116 are defined by structure 130 where a portion of insulative material is removed (e.g., using ablation) to define an area of conductive material. In this way, electrodes 116 are defined by respective portions of structure 130, such as where insulative material is removed to expose conductive material. In other examples, electrodes 116 are individually attached or coupled to structure 130 at one or more fixation points.

In some examples, plurality of electrodes 116 are positioned on structure 130 to generate different electric fields. For example, electrodes can operate as anodes at a first location on structure 130 and cathodes at a second location on structure 130 may be used to generate a first electric field. A second electric field may be generated with electrodes configured to be cathodes proximate the first location on structure 130 and anodes proximate the second location on structure 130. In this manner, alternating between the first and second electric fields may generate electrical current that changes the polarities of cellular components to disrupt cell division. Although a single structure 130 is shown in the example of FIG. 1, two structures, three structures, four structures, five structures, or more structures may be used in different examples. In any case, the combination of structures may provide an overall array of electrodes that can be programmed to deliver alternating electrical fields to a target tissue. These complex electrode geometries can also enable directional sensing that can measure the orientation of electric fields generated in tissue. For example, the system may measure electrical potentials between electrodes at different locations on a lead/structure or between different leads/structures to determine a gradient of electrical potentials and a gradient of the delivered electrical field. The system can then determine electric field spread and configure the electric fields and/or calibrate a predictive model of field spread based on the sensed gradient of electrical potentials.

In some examples, the neurological signals (e.g., an example type of electrical signals) sensed within brain 120 may reflect changes in electrical current produced by the sum of electrical potential differences across brain tissue. Examples of neurological brain signals include, but are not limited to, electrical signals generated from local field potentials (LFP) sensed within one or more regions of brain 120, electroencephalogram (EEG) signals, or electrocorticogram (ECOG) signals. Any of these sensed signals may be intrinsic signals generated by physiological neural activity and/or evoked signals generated in response to a delivered stimulus (e.g., a delivered electrical stimulation signal). It is noted that modulated electric field therapy (e.g., AEF or FTTF therapy) may not evoke neuron propagation or affect other normal neurological function. However, the system may deliver signals intended to affect neurological processes in order to sense signals that may be indicative of physiological states or the response to modulated electric field therapy. In some examples, the system may utilize any electrode combinations to directly sense the electrical field (e.g., field strengths, field locations, or other characteristics) delivered by other electrode combinations. In this manner, the system may confirm expected electrical field strengths, adjust one or more stimulation parameters that define the electrical fields to effect target tissue (e.g., to match a desired stimulation model), and/or adjust the model of stimulation to reflect the reality of tissue characteristics. In some examples, the system may adjust the stimulation parameters defining the electrical fields to accommodate for tissue changes over time and/or lead/structure movement within the patient after surgery or over time. The system may adjust any of these parameters in response to reviewing previously stored data and/or in real-time as sensed data is received or generated.

In some examples, the neurological brain signals that are used to select a stimulation electrode combination may be sensed within the same region of brain 120 as the target tissue site for the electrical stimulation and/or from a region different (e.g., adjacent to or outside of) than the target tissue site. The system may be configured to compute or predict the electrical field at the target tissue based on the signals sensed within the target tissue and/or at a region different than the target tissue. The specific target tissue sites and/or regions within brain 120 may be selected based on the patient condition or location, size, depth, and/or volume of a tumor or resection bed. Thus, due to these differences in target locations, in some examples, the electrodes used for delivering electrical stimulation may be different than the electrodes used for sensing neurological brain signals. In other examples, the same electrodes may be used to deliver electrical stimulation and sense brain signals. However, this configuration of using the same electrodes could require the system to switch between stimulation generation and sensing circuitry and may reduce the time the system can sense brain signals. In some examples, the system may be configured to deliver electrical signals to generate the electrical fields from the same electrode configurations (or using at least some of the same electrodes) in an at least partially interleaved basis.

Electrical stimulation generated by IMD 106 may be configured to manage a variety of disorders and conditions. In some examples, the stimulation generator of IMD 106 is configured to generate and deliver electrical stimulation pulses to patient 112 for AEF or FTTF therapy via electrodes of a selected stimulation electrode combination. However, in other examples, the stimulation generator of IMD 106 may be configured to generate and deliver a continuous wave signal, e.g., a sine wave or triangle wave of specified amplitude (peak to peak) and frequency as part of the electrical fields of the AEF or FTTF therapy. Generally, modulated electric field therapy (e.g., AEF or FTTF therapy) may include the delivery of the continuous wave signal(s), but the waveforms may be symmetric, asymmetric, non-continuous, continuous, cycled, interleaved between different combinations, constant, or otherwise changing over time at random or predetermined sequences. In either case, a stimulation generator within IMD 106 may generate the AEF or FTTF therapy according to a therapy program that is selected at that given time in therapy. In examples in which IMD 106 delivers electrical stimulation in the form of stimulation pulses, a therapy program may include a set of therapy parameter values (e.g., stimulation parameters), such as a stimulation electrode combination for delivering different electrical fields to patient 112, pulse frequency, pulse width, and a current or voltage amplitude of the pulses or continuous signals. As previously indicated, the electrode combination may indicate the specific electrodes 116 that are selected to deliver stimulation signals to tissue of patient 112 and the respective polarities of the selected electrodes. IMD 106 may deliver electrical stimulation intended to contribute to a therapeutic effect. In some examples, IMD 106 may also, or alternatively, deliver electrical stimulation intended to be sensed by other electrode and/or elicit a physiological response, such as an evoked compound action potential (ECAP), that can be sensed by electrodes.

IMD 106 may be implanted within a subcutaneous pocket above the clavicle, or, alternatively, on or within cranium 122 or at any other suitable site within patient 112 such as a lower abdominal or high buttock location. Other configurations might include IMD 106 implanted at multiple locations, such as near a site of tumor occurrence and remote sites of likely tumor transmission or spread. Generally, IMD 106 is constructed of a biocompatible material that resists corrosion and degradation from bodily fluids. IMD 106 may comprise a hermetic housing to substantially enclose components, such as a processor, therapy module, and memory. Other implant locations for IMD 106 may be utilized for treatment of the brain or other tissues. Example alternative implantation sites for IMD 106 may include the lower back, shoulder, neck, abdomen, or any other location.

As shown in FIG. 1, implanted lead extension 110 is coupled to IMD 106 via connector 108 (also referred to as a connector block or a header of IMD 106). In the example of FIG. 1, lead extension 110 traverses from the implant site of IMD 106 and along the neck of patient 112 to cranium 122 of patient 112 to access brain 120. In some examples, one or more structures (such as structure 130) are implanted within the right and/or the left hemispheres of patient 112 in order deliver AEF or FTTF therapy to one or more regions of brain 120, which may be selected based on the patient condition or disorder controlled by therapy system 100. The specific target tissue site and the stimulation electrodes used to deliver stimulation to the target tissue site, however, may be selected, e.g., according to the locations of a tumor or resection bed and/or other sensed patient parameters. Other lead 114, structure 130, and IMD 106 implant sites are contemplated. In some examples, IMD 106 is implanted on or within cranium 122. Structure 130 may be implanted within one hemisphere or span across both hemispheres.

Structure 130 may be positioned to deliver electrical stimulation to one or more target tissue sites within brain 120 (e.g., as an electrode array). One or more of structure 130 may be implanted to position electrodes 116 at desired locations of brain 120 through respective holes, or a common hole, in cranium 122. Structure 130 may be placed at any location within brain 120 such that electrodes 116 are capable of providing electrical stimulation to target tissue sites within brain 120 during treatment. For example, electrodes 116 of structure 130 may be surgically implanted under the dura mater of brain 120 or within the cerebral cortex of brain 120 via a burr hole in cranium 122 of patient 112, and electrically coupled to IMD 106 via lead 114.

In this manner, structure 130 may be designed to be placed surgically in a tumor void or bed to deliver electric fields via an electrode array to cover the interior volume of the debulked void therein. In some examples, structure 130 is configured to expand to fill the volume and deploy electrodes 116 (e.g., structure 130 may hold electrodes 116 with pressure in a target location). Expansion of structure 130 may occur inside patient 112 (e.g., inside cranium 122), or expansion of structure 130 may occur outside of patient 112. Placement of electrodes 116 on structure 130 may define a specific electrode array but may not be otherwise specific to a patient's specific tumor void. However, in some examples, structure 130 is specifically manufactured for a patient's specific tumor void. As each patient may have a unique geometry of a resected tumor void, structure 130 may be configured in such a way to fit a patient's unique geometry, and placement of electrodes 116 may also tailored to a patient's unique geometry. In some examples, structure 130 is configured to be implanted and remain implanted in a patient. However, in some examples, electrodes 116 may be detached from structure 130, and structure 130 does not remain implanted while electrodes 116 remain in the tissue. In examples where electrodes 116 are detached from structure 130, structure 130 may aid in the delivery and placement of electrodes 116.

In some examples, more than one of structure 130 may be implanted in a patient. For example, where a patient has multiple tumor voids or beds, one or more of structure 130 may be placed in each of the multiple tumor voids or beds. In some examples, more than one of structure 130 may be placed in a single tumor void or bed, depending on the shape, size, orientation, and need for stimulation therapy. Where more than one of structure 130 is implanted in a patient, each of structure 130 may be customized to fit the specific shape, size, orientation of each tumor voids or beds.

In the example shown in FIG. 1, IMD 106 includes a memory to store a plurality of therapy programs that each define a set of therapy parameter values. In some examples, IMD 106 may select a therapy program from the memory based on various parameters, such as sensed patient parameters and the identified patient behaviors. IMD 106 may generate electrical stimulation based on the selected therapy program to deliver effective AEF or FTTF therapy that reduces or prevents cancerous cell division or other cellular functions.

External programmer 104 wirelessly communicates with IMD 106 as needed to provide or retrieve therapy information. Programmer 104 is an external computing device that the user, e.g., a clinician and/or patient 112, may use to communicate with IMD 106. For example, programmer 104 may be a clinician programmer that the clinician uses to communicate with IMD 106 and program one or more therapy programs for IMD 106. Alternatively, programmer 104 may be a patient programmer that allows patient 112 to select programs and/or view and modify therapy parameters. The clinician programmer may include more programming features than the patient programmer. In other words, more complex or sensitive tasks may only be allowed by the clinician programmer to prevent an untrained patient from making undesirable changes to IMD 106. IMD 106 may also transmit notifications to programmer 104 for delivery to a user in response to detecting one or more problems with stimulation and/or detection of one or more trigger events for patient 112. Programmer 104 may enter a new programming session for the user to select new stimulation parameters for subsequent therapy. External programmer 104 may display estimated locations of target tissue locations and/or suggested stimulation parameter values for delivering electrical stimulation that affects the target tissue location.

When programmer 104 is configured for use by the clinician, programmer 104 may be used to transmit initial programming information to IMD 106. This initial information may include hardware information, such as the type of lead 114 and the electrode arrangement on structure 130, the position of structure 130 and/or lead 114 within brain 120, the configuration of electrode array 116 on structure 130, initial programs defining therapy parameter values, and any other information the clinician desires to program into IMD 106. Programmer 104 may also be capable of completing functional tests (e.g., measuring the impedance of electrodes 116 or the electric field strength at a strategic location on structure 130). In some examples, programmer 104 may receive sensed signals or representative information and perform the same techniques and functions attributed to IMD 106 herein. In other examples, a remote server (e.g., a standalone server or part of a cloud service) may perform the functions attributed to IMD 106, programmer 104, or any other devices described herein.

Programmer 104 may also be configured for use by patient 112. When configured as a patient programmer, programmer 104 may have limited functionality (compared to a clinician programmer) in order to prevent patient 112 from altering critical functions of IMD 106 or applications that may be detrimental to patient 112. In this manner, programmer 104 may only allow patient 112 to adjust values for certain therapy parameters or set an available range of values for a particular therapy parameter. In one example, a patient programmer may only allow for functions such as turning AEF or FTTF therapy on or off and/or decreasing stimulation intensity. In some examples, programmer 104 may present an indication of delivery time for the patient, such as a screen that indicates the amount of time that the therapy is delivered and/or time the therapy has been off for each day, week, month, etc. For example, programmer 104 may present that “therapy has been delivered for 85% of the time during the last week” or “therapy has been delivered during 6 of the last 7 days.” In addition, programmer 104 may present remaining therapy time available before recharge is required when IMD 106 operates using a rechargeable power source.

Programmer 104 may also provide an indication to patient 112 when therapy is being delivered, when patient input has triggered a change in therapy or when the power source within programmer 104 or IMD 106 needs to be replaced or recharged. For example, programmer 104 may include an alert LED, may flash a message to patient 112 via a programmer display, generate an audible sound or somatosensory cue to confirm patient input was received, e.g., to indicate a patient state or to manually modify a therapy parameter.

Therapy system 100 may be implemented to provide chronic stimulation therapy to patient 112 over the course of several months or years. However, system 100 may also be employed on a trial basis to evaluate therapy before committing to full implantation. If implemented temporarily, some components of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates AEF or FTTF system 100 provides effective treatment to patient 112, the clinician may implant a chronic stimulator within patient 112 for relatively long-term treatment with AEF or FTTF therapy.

Although IMD 106 is described as delivering electrical stimulation therapy to brain 120, IMD 106 may be configured to direct electrical stimulation to other anatomical regions of patient 112 in other examples. In other examples, system 100 may include an implantable drug pump in addition to, or in place of, IMD 106. Further, an IMD may provide other electrical stimulation such as spinal cord stimulation to treat other types of cancer or other diseases or disorders. In some embodiments, the therapy delivered by IMD 106 is designed to enhance the ability of particular drugs to pass through the blood-brain barrier, or is designed to enable particular drugs to pass through the blood-brain barrier. In other embodiments, the therapy delivered by IMD 106 is designed to enhance and/or enable cell membrane permeabilization for the purpose of mediating cell transfection or enhancing viral delivery to target cells. By being designed to achieve these goals, IMD 106 may be configured (via specific stimulation parameter values) to deliver electrical field therapy that increases blood-brain barrier permeabilization and/or enhances cell membrane permeabilization.

According to the techniques of the disclosure, system 100 may include processing circuitry configured to receive a request to deliver alternating electric field (AEF) or focused tumor treating field (FTTF) therapy, determine therapy parameter values that define the AEF or FTTF therapy, wherein the AEF or FTTF therapy includes delivery of a first electric field and a second electric field, control IMD 106 to deliver the first electric field from a first electrode combination of implanted electrodes, and control IMD 106 to deliver, alternating with the first electric field, the second electric field from a second electrode combination of implanted electrodes different than the first electrode combination. The request may be via user input and/or an automated system request to start AEF or FTTF therapy delivery.

The electrical fields that IMD 106 alternates over time to produce the AEF or FTTF therapy may involve different electrode combinations and/or different methods for alternating the electrical fields between different electrode combinations (e.g., different electrodes and/or different polarities of the same or different electrodes). In one example, the first electrode combination includes a first set of electrodes defined as cathodes and a second set of electrodes defined as anodes, and the second electrode combination includes the first set of electrodes defined as anodes and the second set of electrodes defined as cathodes.

IMD 106 may generally use the same pulse or signal frequency for generating electrical fields of the AEF or FTTF therapy. In one example, the frequency may be approximately 150 kHz. In another example, the frequency may be approximately 200 kHz. In general, the frequency may be selected from a range of approximately 100 kHz through 300 kHz, but frequencies higher or lower than this range may be used in other examples. In some examples, the frequencies employed by IMD 106 are selected based on the types of cells targeted for treatment. For example, if targeting cancer cells of a certain size (e.g., 13 micrometers in diameter), the IMD 106 delivers therapy with a frequency (e.g., 200 kHz) at which therapy will be more effective for that cell size. In some examples, the frequency or range of frequencies at which the electrical fields are delivered may be selected based on a workup of a patient biopsy or based on a lookup table according to the tumor type and associated distributions of cell sizes. In some examples, the minimum or maximum frequencies may be selected in order to avoid affecting sizes of healthy cells within the electric fields that may differ from the size of the tumor cells.

IMD 106 may alternate the electrical fields in AEF or FTTF therapy by delivering the electrical fields from different electrodes and/or electrodes with different polarities. In one example, IMD 106 may continually shift the polarities of the electrodes in one direction with respect to the electrode array. The first electrode combination may include a first set of electrodes defined as cathodes and a second set of electrodes defined as anodes, the second electrode combination may include a third set of electrodes defined as anodes and a fourth set of electrodes defined as cathodes

As shown in FIG. 1, electrodes 116 (e.g., at least two electrodes) used to deliver the AEF or FTTF therapy are disposed on a structure 130 placed adjacent a resection bed of tissue. In some examples, at least two electrodes of the implanted electrodes 116 used to deliver AEF or FTTF therapy are subcutaneous electrodes (e.g., electrodes implanted beneath the skin and superficial of the bone). In some examples, one or more electrodes may be implanted on the inside of the removed skull portion during surgery in order to provide a relatively easy implantation of electrodes within the skull but outside of the brain. In other examples, a system may deliver the AEF or FTTF therapy using a plurality of external cutaneous electrodes in combination with a plurality of implanted electrodes 116. For example, a first electrical field of the AEF or FTTF therapy may be delivered between two or more external electrodes and the second electrical field of the AEF or FTTF therapy is delivered between two or more implanted electrodes 116. In other example, two or more electrical fields that create the AEF or FTTF therapy each utilize one or more external electrodes. In some examples, the current passes between only external electrodes or between only implanted electrodes. In other examples, one or more electrical fields may be created between any combination of external and implanted electrodes 116. An external device may deliver current to the external electrodes, either independent controlled or controlled based on communication between the external device and IMD 106.

Generally, AEF or FTTF therapy is described herein as a treatment to already present tumors, such as glioblastomas. In other examples, the application of AEF or FTTF therapy can reduce the extent of metastatic tumor burden and seeding of tumors from a remote tumor source. Therefore, AEF or FTTF treatment could be delivered to protect tissue regions from metastatic spread. For example, AEF or FTTF could be utilized to provide global brain protection in the setting of a known malignant tumor within the body, particularly those that have a propensity for cerebral dissemination (e.g., Melanoma). AEF or FTTF could be delivered to prevent additional metastatic spread of tumor within the organ system of current metastatic dissemination. In addition, AEF or FTTF implant planning could be provided for the protection of certain neurological function (e.g., motor function), such that the implant system 100 would be focused on treatment to the pre-central gyrus and/or corticospinal tract to preserve its function and avoid seeding.

An AEF or FTTF delivery implant (e.g., IMD 106, lead 114, and structure 130) could be utilized to prophylactically treat a body region that is expected to have a high risk for metastatic dissemination (e.g., a presumed location where tumor would progress next). An example of this is the axillary lymph nodes in the setting of a newly diagnosed breast cancer. Lymphatic channels have predictable flow and are common highways for metastatic dissemination. Therefore, implantation strategies that focus on the systematic treatment of these highways could meaningfully impact the propensity and capability for tumors to metastatically spread.

AEF or FTTF delivery could also utilize electrodes within arteries to permit wider control of metastatic spread. Put another way, AEF or FTTF delivery could reduce the likelihood of metastatic tumor cells to exit the blood stream and seed other regions within the body. In one example, treatment of the carotid arteries could reduce the ability for tumor cells to exit the bloodstream and invade the cerebral tissue. AEF or FTTF delivery within the arteries could provide advantageous pharmacokinetic impacts on drugs within the tumor region and therefore justify intra-arterial placement of electrodes for AEF or FTTF delivery. In this manner, AEF or FTTF therapy described herein may be applied to numerous different tissues and for numerous different reasons. For example, AEF or FTTF therapy or other modulated electric field therapy may be provided as a preventative therapy to tissue not yet diagnosed as cancerous but at risk for tumor occurrence based on one or more characteristics such as genetic markers, environmental factors, or any other risk factors.

The architecture of system 100 illustrated in FIG. 1 is shown as an example. The techniques as set forth in this disclosure may be implemented in the example system 100 of FIG. 1, as well as other types of systems not described specifically herein. Nothing in this disclosure should be construed so as to limit the techniques of this disclosure to the example architecture illustrated by FIG. 1.

System 100 is generally described as including IMD 106 and external programmer 104. However, in other examples, an external medical device may be configured to perform any of the techniques described herein or described with respect to IMD 106. The external medical device may be coupled to percutaneous leads or other devices that pass through the skin in order to dispose implanted electrodes at various locations within patient for at least partially delivering electric field therapy and/or sensing signals as described herein. Additionally, or alternatively, the external device may be coupled to external electrodes configured to at least partially deliver electric field therapy and/or sense signals as described herein. The external medical device may be configured to communicate with programmer 104 and/or partially or fully incorporate structures to perform the various functionality described with respect to programmer 104.

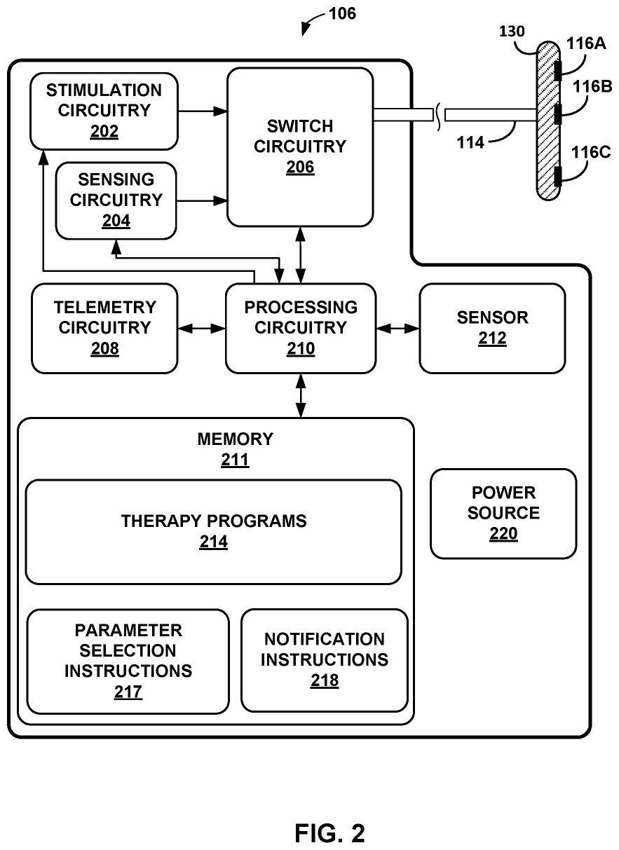

FIG. 2 is a block diagram of the example IMD of FIG. 1 for delivering therapy according to an example of the techniques of the disclosure. In the example shown in FIG. 2, IMD 106 includes processing circuitry 210, memory 211, stimulation circuitry 202, sensing module 204, switch module 206, telemetry module 208, sensor 212, and power source 220. In some examples, any or all of processing circuitry 210, memory 211, stimulation circuitry 202, sensing module 204, switch module 206, telemetry module 208, sensor 212, and power source 220 is included in structure 130, and structure 130 may be configured to perform any of the function of IMD 106 described herein. Each of these modules may be or include electrical circuitry configured to perform the functions attributed to each respective module. For example, processing circuitry 210 may include processing circuitry, switch module 206 may include switch circuitry, sensing module 204 may include sensing circuitry, and telemetry module 208 may include telemetry circuitry. Switch module 206 may not be used for multiple current source and sink configurations, but one or more switches may still be used to disconnect sensing module 204 from the source and sinks in such a configuration. Memory 211 may include any volatile or non-volatile media, such as a random-access memory (RAM), read only memory (ROM), non-volatile RAM (NVRAM), electrically erasable programmable ROM (EEPROM), flash memory, and the like. Memory 211 may store computer-readable instructions that, when executed by processing circuitry 210, cause IMD 106 to perform various functions. Memory 211 may be a storage device or other non-transitory medium.

In the example shown in FIG. 2, memory 211 stores therapy programs 214 that include respective stimulation parameter sets that define AEF or FTTF therapy. Each stored therapy program 214 defines a particular set of electrical stimulation parameters (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated.

Memory 211 may also include parameter selection instructions 217 and notification instructions 218. Parameter selection instructions 217 may include instructions that control processing circuitry 210 selecting different stimulation parameter values such as electrode combinations, amplitudes, pulse frequencies, or other parameter values for compensating for various locations of target tissue or feedback related to changes in patient condition or tissue state. Parameter selection instructions 217 may include instructions for processing circuitry 210 to select parameter values based on various feedback variables. Notification instructions 218 may define instructions that control processing circuitry 210 actions such as transmitting an alert or other notification to an external device, such as programmer 104, that therapy is on or off, or if changes to AEF or FTTF therapy have been made or are recommended.

In some examples, the sense and stimulation electrode combinations may include the same subset of electrodes 116, a housing of IMD 106 functioning as an electrode, or may include different subsets or combinations of such electrodes. Thus, memory 211 can store a plurality of sense electrode combinations and, for each sense electrode combination, store information identifying the stimulation electrode combination that is associated with the respective sense electrode combination. The associations between sense and stimulation electrode combinations can be determined, e.g., by a clinician or automatically by processing circuitry 210. In some examples, corresponding sense and stimulation electrode combinations may comprise some or all of the same electrodes. In other examples, however, some or all of the electrodes in corresponding sense and stimulation electrode combinations may be different. For example, a stimulation electrode combination may include more electrodes than the corresponding sense electrode combination in order to increase the efficacy of the AEF or FTTF therapy.

Stimulation circuitry 202, under the control of processing circuitry 210, generates stimulation signals for delivery to patient 112 via selected combinations of electrodes 116. An example range of electrical stimulation parameters believed to be effective in AEF or FTTF therapy to manage cellular activity include:
1. Frequency (e.g., waveform frequency or pulse rate): between approximately 50 kHz and approximately 500 kHz, such as between approximately 100 kHz to 300 kHz, or such as approximately 150 kHz or 200 kHz.
2. In the case of a voltage-controlled system, Voltage Amplitude: between approximately 0.1 volts and approximately 50 volts, such as between approximately 2 volts and approximately 10 volts.
3. In the alternative case of a current controlled system, Current Amplitude: between approximately 0.2 milliamps to approximately 100 milliamps, such as between approximately 1.3 milliamps and approximately 2.0 milliamps.
4. Pulse Width: between approximately 1 microseconds and approximately 10 microseconds, such as between approximately 1 microseconds and approximately 5 microseconds, or between approximately 2 microseconds and approximately 10 microseconds.
5. Cycle time (e.g., communication time), which is the time a waveform remains consistent before switching off or switching to a new waveform. The cycle time may be selected from a range of 30 seconds and 30 minutes, or within a range from 1 minute to 10 minutes. Shorter and longer cycle times may be used in other examples.

Accordingly, in some examples, stimulation circuitry 202 generates electrical stimulation signals in accordance with the electrical stimulation parameters noted above. Other ranges of therapy parameter values may also be useful, and may depend on the target stimulation site within patient 112. While stimulation pulses are described, stimulation signals may be of any form, such as continuous-time signals (e.g., sine waves) or the like. Stimulation signals configured to elicit ECAPs or other evoked physiological signals may be similar or different from the above parameter value ranges. In addition, sensing circuitry 204 may be configured to sense signals via one or more electrode combinations on one or more lead 114 (e.g., the same or different electrodes may deliver stimulation and sense electrical signals).

Processing circuitry 210 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 may control stimulation circuitry 202 according to therapy programs 214 stored in memory 211 to apply particular stimulation parameter values specified by one or more of programs, such as voltage amplitude or current amplitude, pulse width, or pulse rate.

In the example shown in FIG. 2, the set of electrodes 116 includes electrodes 116A, 116B, and 116C. Processing circuitry 210 also controls switch module 206 to apply the stimulation signals generated by stimulation circuitry 202 to selected combinations of electrodes 116. In particular, switch module 206 may couple stimulation signals to selected conductors within lead 114 or structure 130, which, in turn, deliver the stimulation signals across selected electrodes 116. Switch module 206 may be a switch array, switch matrix, multiplexer, or any other type of switching module configured to selectively couple stimulation energy to selected electrodes 116 and to selectively sense neurological brain signals with selected electrodes 116. Hence, stimulation circuitry 202 is coupled to electrodes 116 via switch module 206 and conductors within lead 114 and/or structure 130. In some examples, however, IMD 106 does not include switch module 206, such as if each electrode is assigned a respective current and sink (e.g., independent current source).

Stimulation circuitry 202 may be a single channel or multi-channel stimulation generator. In particular, stimulation circuitry 202 may be capable of delivering a single stimulation pulse, multiple stimulation pulses, or a continuous signal at a given time via a single electrode combination or multiple stimulation pulses at a given time via multiple electrode combinations. In some examples, however, stimulation circuitry 202 and switch module 206 may be configured to deliver multiple channels on a time-interleaved basis. For example, switch module 206 may serve to time divide the output of stimulation circuitry 202 across different electrode combinations at different times to deliver multiple programs or channels of stimulation energy to patient 112 (e.g., cycling between regimes of stimulation on a fixed or variable sequence). Alternatively, stimulation circuitry 202 may comprise multiple voltage or current sources and sinks that are coupled to respective electrodes to drive the electrodes as cathodes or anodes. In this example, IMD 106 may not require the functionality of switch module 206 for time-interleaved multiplexing of stimulation via different electrodes.

Although sensing module 204 is incorporated into a common housing with stimulation circuitry 202 and processing circuitry 210 in FIG. 2, in other examples, sensing module 204 may be in a separate housing from IMD 106 and may communicate with processing circuitry 210 via wired or wireless communication techniques. Example neurological brain signals include, but are not limited to, a signal generated from local field potentials (LFPs) within one or more regions of brain 120. EEG and ECOG signals are examples of other types of electrical signals that may be measured within brain 120. Other examples include sensed signals representative of electric field or voltage gradients caused by a remote electrode as recorded by a proximal electrode or electrode pair. Instead of, or in addition to, LFPs, IMD 106 may be configured to detect patterns of single-unit activity and/or multi-unit activity. IMD 106 may sample this activity at rates above 1,000 Hz, and in some examples within a frequency range of 6,000 Hz to 500,000 Hz. IMD 106 may identify the wave-shape of single units and/or an envelope of unit modulation that may be features used to differentiate or rank electrodes. In some examples, this technique may include phase-amplitude coupling to the envelope or to specific frequency bands in the LFPs sensed from the same or different electrodes. In some examples, the sampling technique may be set to identify the electric field strength at any location. For example, IMD 106 may include a peak following circuitry that holds the amplitude of a field of a specific frequency for later sampling. Alternatively, the response of a resonant circuit may be tuned to the AEF or FTTF frequency might sampled to infer the field strength of the desired signal.

Sensor 212 may include one or more sensing elements that sense values of a respective patient parameter, such as patient activity (e.g., movement and/or sleep). For example, sensor 212 may include one or more accelerometers, optical sensors, chemical sensors, temperature sensors, pressure sensors, or any other types of sensors. Sensor 212 may output patient parameter values that may be used as feedback to control delivery of AEF or FTTF therapy. IMD 106 may include additional sensors within the housing of IMD 106 and/or coupled via lead 114 and/or structure 130. In addition, IMD 106 may receive sensor signals wirelessly from remote sensors via telemetry module 208, for example. In some examples, one or more of these remote sensors may be external to patient (e.g., carried on the external surface of the skin, attached to clothing, or otherwise positioned external to the patient).

Telemetry module 208 supports wireless communication between IMD 106 and an external programmer 104 or another computing device under the control of processing circuitry 210. Processing circuitry 210 of IMD 106 may receive, as updates to programs, values for various stimulation parameters such as magnitude and electrode combination, from programmer 104 via telemetry module 208. The updates to the therapy programs may be stored within therapy programs 214 portion of memory 211. In addition, processing circuitry 210 may control telemetry module 208 to transmit alerts or other information to programmer 104 that indicate a lead moved with respect to tissue. Telemetry module 208 in IMD 106, as well as telemetry modules in other devices and systems described herein, such as programmer 104, may accomplish communication by radiofrequency (RF) communication techniques. In addition, telemetry module 208 may communicate with external medical device programmer 104 via proximal inductive interaction of IMD 106 with programmer 104. Accordingly, telemetry module 208 may send information to external programmer 104 on a continuous basis, at periodic intervals, or upon request from IMD 106 or programmer 104.

Power source 220 delivers operating power to various components of IMD 106. Power source 220 may include a small rechargeable or non-rechargeable battery and a power generation circuit to produce the operating power. Recharging may be accomplished through proximal inductive interaction between an external charger and an inductive charging coil within IMD 106. In some examples, power requirements may be small enough to allow IMD 106 to utilize patient motion and implement a kinetic energy-scavenging device to trickle charge a rechargeable battery. In other examples, traditional batteries may be used for a limited period of time. In other examples, IMD 106 may include a power receiving antenna a corresponding circuitry to continually receive external power that enables IMD 106 to deliver electric field therapy indefinitely without possible internal power source drain.

According to the techniques of the disclosure, processing circuitry 210 of IMD 106 delivers, via electrodes 116 disposed on structure 130, electrical stimulation therapy to patient 112. The AEF or FTTF therapy is defined by one or more therapy programs 214 having one or more parameters stored within memory 211. For example, the one or more parameters include a current amplitude (for a current-controlled system) or a voltage amplitude (for a voltage-controlled system), a pulse rate or frequency, and a pulse width, or quantity of pulses per cycle. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time.

One or more features determined from the sensed electrical signals may include or represent different characteristics of the sensed electrical signals. Example features of the sensed electrical signals may include voltage (peak or average), orientation of resulting electric field, impedance, spectral power, one or more frequencies, one or more frequency bands, or any other characteristics of the sensed signals. In this manner, the one or more features may be features in the time domain, frequency domain, or any other signal domain relevant to identify the sensed signals. For example, processing circuitry 210 may be configured to determine the one or more features by at least determining a sensed voltage for each electrode combination of the plurality of electrode combinations. In some examples, processing circuitry 210 may be configured to determine the one or more features by at least determining at least one of a power, a frequency band, a time domain feature, and/or a frequency domain feature of each signal of the plurality of signals from respective electrode combinations of the plurality of electrode combinations.

Processing circuitry 210 may be configured to control a user interface to display various elements and aspects related to the planning and delivery of AEF or FTTF therapy. For example, processing circuitry 210 may transmit sensed data related to AEF or FTTF therapy such as impedance tomography, stimulation parameters, patent activity, and any other data to programmer 104 for display to the user via the user interface. In some examples, processing circuitry 210 may be configured to receive user selection of a value for one or more stimulation parameters that at least partially defines AEF or FTTF therapy. For example, the user interface may present a visual indication of the estimated location of the target tissue and electrical field strength variations over different locations of the target tissue.

In some examples, any or all components of IMD 106 previously discussed may be contained within a deployable auxetic structure, such as structure 130 of FIG. 1. For example, structure 130 may contain processing circuitry to control and delivery electrical stimulation therapy and not need to be wired to another device. In some examples where structure 130 is able to delivery therapy by itself, structure 130 may contain a power source (e.g., a battery). However, in some examples, structure 130 is able to wirelessly receive energy from a device external to patient 112 and transfer electrical stimulation energy without a power source (e.g., a battery-less energy transmission, such as an ultrasound or radiofrequency source).

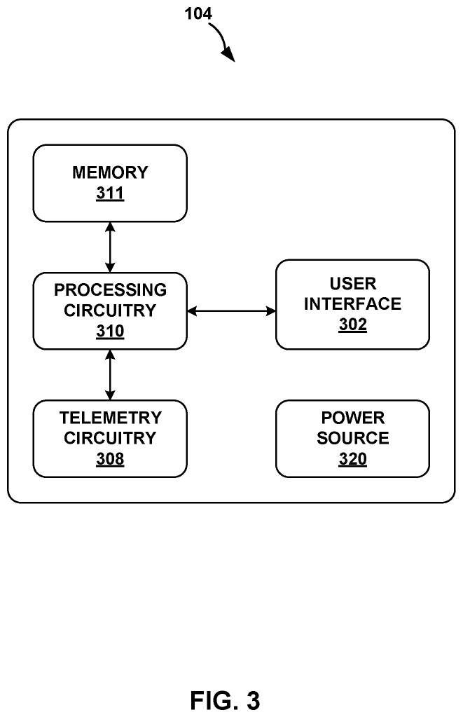

FIG. 3 is a block diagram of the external programmer of FIG. 1 for controlling delivery of therapy according to an example of the techniques of the disclosure. Although programmer 104 may generally be described as a hand-held device, programmer 104 may be a larger portable device or a more stationary device. In some examples, programmer 104 may be referred to as a tablet computing device or a smart phone computing device. In addition, in other examples, programmer 104 may be included as part of a bed-side monitor, an external charging device or include the functionality of an external charging device. As illustrated in FIG. 3, programmer 104 may include a processing circuitry 310, memory 311, user interface 302, telemetry module 308, and power source 320. Memory 311 may store instructions that, when executed by processing circuitry 310, cause processing circuitry 310 and external programmer 104 to provide the functionality ascribed to external programmer 104 throughout this disclosure. Each of these components, or modules, may include electrical circuitry that is configured to perform some or all of the functionality described herein. For example, processing circuitry 310 may include processing circuitry configured to perform the processes discussed with respect to processing circuitry 310.

In general, programmer 104 comprises any suitable arrangement of hardware, alone or in combination with software and/or firmware, to perform the techniques attributed to programmer 104, and processing circuitry 310, user interface 302, and telemetry module 308 of programmer 104. In various examples, programmer 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. Programmer 104 also, in various examples, may include a memory 311, such as RAM, ROM, PROM, EPROM, EEPROM, flash memory, a hard disk, a CD-ROM, comprising executable instructions for causing the one or more processors to perform the actions attributed to them. Moreover, although processing circuitry 310 and telemetry module 308 are described as separate modules, in some examples, processing circuitry 310 and telemetry module 308 may be functionally integrated with one another. In some examples, processing circuitry 310 and telemetry module 308 correspond to individual hardware units, such as ASICs, DSPs, FPGAs, or other hardware units.

Memory 311 (e.g., a storage device) may store instructions that, when executed by processing circuitry 310, cause processing circuitry 310 and programmer 104 to provide the functionality ascribed to programmer 104 throughout this disclosure. For example, memory 311 may include instructions that cause processing circuitry 310 to obtain a parameter set from memory, present a model of patient anatomy for predicting electrical field strengths, provide an interface that recommends or otherwise facilitates parameter value selection, or receive a user input and send a corresponding command to IMD 106, or instructions for any other functionality. In addition, memory 311 may include a plurality of programs, where each program includes a parameter set that defines stimulation therapy.

User interface 302 may include a button or keypad, lights, a speaker for voice commands, a display, such as a liquid crystal (LCD), light-emitting diode (LED), or organic light-emitting diode (OLED). In some examples the display may be a presence-sensitive screen, such as a touch screen. User interface 302 may be configured to display any information related to the delivery of stimulation therapy, detected trigger events, progression of therapy, suggested stimulation parameter values, sensed patient parameter values, or any other such information. User interface 302 may also receive user input via user interface 302. The input may be, for example, in the form of pressing a button on a keypad or selecting an icon from a touch screen.

Telemetry module 308 may support wireless communication between IMD 106 and programmer 104 under the control of processing circuitry 310. Telemetry module 308 may also be configured to communicate with another computing device via wireless communication techniques, or direct communication through a wired connection. In some examples, telemetry module 308 provides wireless communication via an RF or proximal inductive medium. In some examples, telemetry module 308 includes an antenna, which may take on a variety of forms, such as an internal or external antenna. In some examples, IMD 106 and/or programmer 104 may communicate with remote servers via one or more cloud-services in order to deliver and/or receive information between a clinic and/or programmer.

Examples of local wireless communication techniques that may be employed to facilitate communication between programmer 104 and IMD 106 include RF communication according to the 802.11 or Bluetooth specification sets or other standard or proprietary telemetry protocols. Security protocols and encryption techniques may be applied to enhance the security of the communication techniques. In addition, other external devices may be capable of communicating with programmer 104 without needing to establish a secure wireless connection. As described herein, telemetry module 308 may be configured to transmit a spatial electrode movement pattern or other stimulation parameter values to IMD 106 for delivery of stimulation therapy.

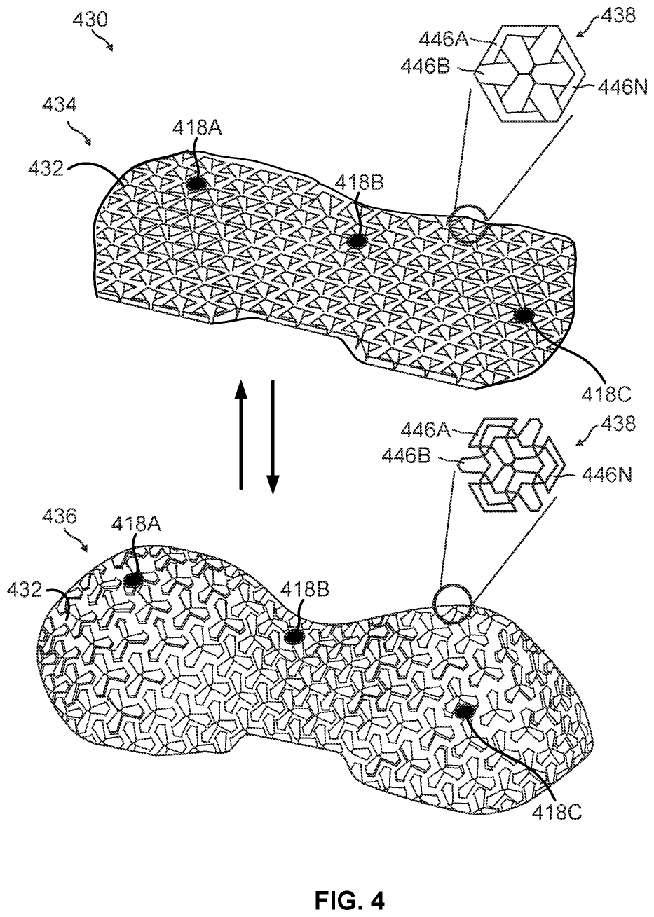

FIG. 4 illustrates a transformable auxetic structure 430. Structure 430 may be an example of structure 130 described in connection with FIG. 1. Structure 430 may define a major surface 432. Structure 430 includes a pattern of repeating unit cells 438 (e.g., auxetic unit cells) which form major surface 432. Unit cells 438 may be configured to expand in response to a stimulus (e.g., a directed force, such as tension force or compression force applied to structure 430). For example, a tensile force or tension force applied to certain areas (e.g., opposing ends of major surface 432 of structure 430) may cause structure 430 to expand into a non-planar configuration. Unit cells 438 may be configured to convert structure 430 between a compact configuration 434 (that is of a substantially simpler form factor such as sheet, cylinder, or other shape) and an expanded configuration 436 (that has one or more curved portions in 3D). In the compact configuration, structure 430 may be substantially flat or 2D. Structure 430 may carry one or more electrodes or one or more leads via a plurality of fixation points 418 (shown individually in the example of FIG. 4 as 418A, 418B, and 418C) (also referred to herein as fixation points 418 or one or more fixation points 418).

In the example of FIG. 4, structure 430 is transformable between compact configuration 434 and expanded configuration 436. In some examples, in compact configuration 434, which may be a first configuration, structure 430 is substantially flat (e.g., flat or substantially flat to the extent permitted by manufacturing tolerances or to account for minor variations). In some examples, substantially flat may mean that structure 430 rises to a height less than 5 percent of the length of structure 430 over the length of structure 430 (e.g., a curvature less than 5 mm for a length of 100 mm). For example, in compact configuration 434, major surface 432 resides in a two-dimensional plane such that major surface 432 is substantially flat and does not have any three-dimensional contours extending from major surface 432. In some examples, when structure 430 is in compact configuration 434, unit cells 438 are collapsed such that major surface 432 is substantially flat. While the example of FIG. 4 represents the first configuration as being a configuration in which structure 430 is substantially flat, the first configuration may include other configurations, including, but not limited to a flat or substantially flat sheet, a cylinder (e.g., a cylinder with a uniform lateral surface), or other simple form factors.

In some examples, in expanded configuration 436, which may be a second configuration, structure 430 defines one or more three-dimensional contours. For example, in expanded configuration 436, major surface 432 is deployed to conform to a three-dimensional contour (e.g., the three-dimensional contour of a resected tumor). In some examples, when structure 430 is in expanded configuration 436, unit cells 438 are expanded such that major surface 432 resides in a three-dimensional space, and does not have a major surface that is substantially flat.

In some examples, structure 430 is a bi-stable auxetic structure in which structure 430 may reside in either compact configuration 434 (e.g., a first stable configuration) or expanded configuration 436 (e.g., a second stable configuration) without any additional forces holding structure 430 in the respective configuration. For example, when a first stimulus is provided to structure 430 (e.g., a first directed force, such as tension force or compression force provided to either end of structure 430) to transform structure 430 from compact configuration 434 to expanded configuration 436, structure 430 will remain in expanded configuration 436 even when the first stimulus is removed. Similarly, when a second stimulus is provided to structure 430 (e.g., a second directed force, such as tension force or compression force provided to either end of structure 430) to transform structure 430 from expanded configuration 436 back to compact configuration 434, structure 430 will remain in compact configuration 434 even when second stimulus is removed. In some examples, structure 430 is unstable in configurations between compact configuration 434 (e.g., the first stable configuration) and expanded configuration 436 (e.g., a second stable configuration). For example, in intermediate configurations between compact configuration 434 and expanded configuration 436, structure 430 will tend to transform to either compact configuration 434 or expanded configuration 436 (e.g., depending on whether the intermediate configuration is closer to compact configuration 434 or expanded configuration 436). In other examples, structure 430 is not bi-stable, and requires a force to hold structure 430 in expanded configuration 436 (e.g., a balloon, heat set, or other type of force or treatment to hold structure 430 in expanded configuration 436).

In the example of FIG. 4, unit cells 438 include plurality of elements 446A, 446B, and 446N (hereinafter collectively referred to as plurality of elements 446) configured to move or transform such that structure 430 transforms between compact configuration 434 and expanded configuration 436. For example, each unit cell of unit cells 438 includes plurality of elements 446 having increased distances between adjacent elements of plurality of elements 446 in expanded configuration 436 as compared to compact configuration 434. In compact configuration 434, each element of plurality of elements 446 may be touching or nearly touching adjacent elements. When a force (e.g., a directed force, such as tension force or compression force) is applied to each unit cell of unit cells 438, each element of plurality of elements 446 may separate from adjacent elements of plurality of elements 446 and/or from a center or references point of each unit cell of unit cells 438. In some examples, each element of plurality of elements 446 moves uniformly in relation to other elements of plurality of elements 446 during transformation from compact configuration 434 to expanded configuration 436. However, in some examples each element of plurality of elements 446 moves differently in relation to other elements of plurality of elements 446 during transformation from compact configuration 434 to expanded configuration 436.

In some examples, each unit cell of unit cells 438 may be unique and create a specific contour (e.g., three-dimensional contour) when expended such that when each unit cell of unit cells 438 is expanded, structure 430 defines a specific three-dimensional contour (e.g., pre-determined contour) in expanded configuration 436. The individual configuration, orientation, and arrangement of each unit cells of unit cells 438 in relation to each other during manufacturing may determine the ultimate expanded three-dimensional contour of structure 430, which may correspond to a specific contour of a patient anatomy (e.g., a contour of a resected tumor bed of a patient). However, in some examples, the specific three-dimensional contour of structure 430 in expanded configuration 436 may not be patient specific, but rather fit a general or common shape for a given therapy application. In some examples, the general shape of structure 430 may not be patient specific, but the shape of structure 430 can be further refined to specific contour of a patient anatomy.

In the example of FIG. 4, structure 430 is manufactured (e.g., where unit cells 438 are formed from surface 432) in compact configuration 434 (e.g., where structure is 430 is substantially flat and/or surface 432 does not define any three-dimensional contours). Manufacturing in compact configuration 434 may reduce complexity and physical space needed for manufacturing, and may increase accuracy and reproducibility of structures such as structure 430. For example, multiple structures, such as structure 430, can be mass-produced in a substantially flat orientation and further stored until deployed to an expanded configuration where structure defines one or more three-dimensional contours (e.g., before or during surgery when structure 430 is placed in patient 112). During the manufacturing process, a series of incisions (or in some examples, a series of scores, gouges, grooves, impressions, etc.) are made into major surface 432 to create unit cells 438. The incisions that define each unit cell of unit cells 438 may be made by any suitable process, include physical cutting, etching, chemical material removal, laser cutting, or the like. In some examples, when incisions are made during the manufacturing process, material may also be removed from structure 430 such that each unit cell of unit cells 438 defines one or more gaps (e.g., voids of material) within each until cell. Incisions into unit cells 438 may define plurality of elements 446.

In some examples, unit cells 438 may be shaped and/or arranged (e.g., in a pattern of repeating unit cells) such that each unit cell of unit cells 438 is connected to one or more adjacent unit cells. In the example of FIG. 4, when structure 430 is in compact configuration 434, each unit cells of unit cells 438 defines a hexagon (e.g., the outer edges of each auxetic unit cells collectively define a hexagon), and each unit cell of unit cells 438 is connected to adjacent auxetic unit cells at each edge of the hexagon. While each unit cell of unit cells 438 is shown as a hexagon, each unit cell of unit cells 438 may define any suitable shape for unit cells (triangle, square, rectangle, pentagon, irregular shapes, etc.). In some examples, each unit cells of unit cells 438 define the same outer shape and size (but may differ by inner incisions within each auxetic unit cell). In some examples, unit cells 438 may define a repeating pattern. In some examples, each unit cell of unit cells 438 may define different outer shapes and sizes compared to other unit cells of unit cells 438.

In some examples, expanded configuration 436 of structure 430 includes a contour associated with one or more pre-determined target locations for therapy within a patient. For example, a generic mapping or a patient-specific mapping of a resected tumor bed (e.g., from a computerized tomography (CT) scan) may be provided during the manufacturing process of structure 430, such that unit cells 438 are created to correspond to a three-dimensional contour of structure 430 that maps or otherwise fits the patient-specific mapping. Further, each of plurality of fixation points 418 for a plurality of electrodes or a plurality of leads may correspond to one or more pre-determined target location for placement of one or more electrodes or one or more leads that carry one or more electrodes. When one or more electrodes are coupled to, disposed on, or carried by structure 430, the locations of the electrodes on structure 430 may corresponding to one or more pre-determined target locations within a patient for therapy delivery (e.g., such as when structure 430 is in expanded configuration 436). While structure 430 is primarily described in relation to target locations in a resected tumor bed in patient 112, other target locations for therapy delivery of structure 430 may be possible (e.g., wrapped around a nerve or nerve bundle via a cylindrically shaped structure). Additionally, while structure 430 is primarily described in relation to target locations located within brain 120 of patient 112, other suitable locations are possible (e.g., within soft tissue, bone, organs, etc.).

Structure 430 may be sized according to the target location of the brain and the type of therapy being delivery. For example, structure 430 may include a thickness of approximately 1 millimeter (e.g., between 0.5 and 1.5 millimeters), and have a uniform thickness throughout. In some examples, structure 430 may vary in thickness. In some examples, a thickness of structure 430 may change (e.g., increase or decrease) when structure 430 is deployed or transformed from compact configuration 434 to expanded configuration 436.

In the example of FIG. 4, structure 430 may be a non-conductive material, such as a polymer, ceramic, or a non-conductive metal. In some examples, structure 430 may include a flexible material configured to conform to one or more patient anatomical features (e.g., the geometry of a resected tumor bed). However, in some examples, structure 430 may include a stiff material, but structure 430 may nonetheless conform to one or more patient anatomical features as the three-dimensional contours of structure 430 may allow flexibility at joints within unit cells 438. In other examples, structure 430 may be formed of an electrically conductive material that can conduct current to tissue. In some examples, portions of structure 430 may be covered (e.g., coated) with an electrically insulative material while other portions of structure 430 are exposed to conduct current to tissue at those exposed portions.

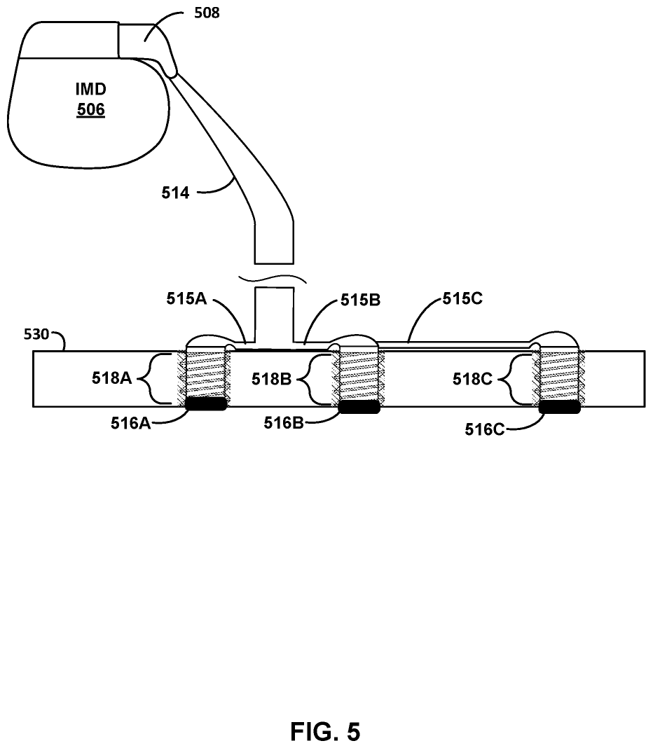

FIG. 5 illustrates another example structure 530, which may be an expandable auxetic structure, including a plurality of fixation points 518 (shown individually as fixation point 518A, fixation point 518B, and fixation point 518C) for coupling a plurality of electrodes 516 (shown individually as electrode 516A, electrode 516B, and electrode 516C) and/or a plurality of conductors 515 (shown individually as conductor 515A, conductor 515B, and conductor 515C) to structure 530. Structure 530 may be an example of structure 130 or structure 430 shown in connection with FIG. 1 or FIG. 4 respectively. In the example of FIG. 5, lead 514 is coupled to an IMD 506 via a connector 508. Lead 514 extends from IMD 506 to structure 530 and lead 514 splits into plurality of conductors 515 (e.g., lead 514 splits into conductor 515A, conductor 515B, and conductor 515C). Plurality of conductors 515 may be disposed on a surface of structure 530 (e.g., a surface opposite of electrodes 516) or within structure 530 (e.g., between major surfaces of structure 530). Plurality of conductors 515 may be electrically coupled to electrodes 516 and/or lead 514. Plurality of conductors 515 may be fed through respective plurality of fixation points 518. Additionally or alternatively, a lead, such as lead 514, which is coupled to an IMD 506 via a connector 508, is connected directly to structure 530.

In some examples, structure 530 includes a conductive material that may facilitate transfer electrical energy from lead 514 to plurality of electrodes 516 without needing plurality of electrodes 516 to be coupled to or carried by lead 514 or plurality of conductors 515. In examples where structure 530 facilities transfer of electrical energy from lead 514 to plurality of electrodes 516, structure 530 may include a conductive material. In some examples, a majority of structure 530 includes a conductive material (e.g., a conductive metal). However, in some examples, structure 530 includes conductive channels (e.g., wires) throughout structure 530 to facilitate transfer of electrical energy from lead 514 to plurality of electrodes 516.

While the example of FIG. 5 only illustrates one lead 514 connecting plurality of electrodes 516 and/or plurality of conductors 515 to IMD 506, multiple leads may be attached to structure 530 (e.g., to serve as a redundant electrical connection, or to deliver electrical energy to different areas of structure 530). Similarly, although plurality of electrodes 516 is only shown as three total electrodes, plurality of electrodes 516 can include any number or combination of electrodes. For example, structure 530 can carry any suitable number of electrodes (e.g., one, two, three, four, or more electrodes depending on the target region for therapy and the type of therapy being delivered).

In the example of FIG. 5, plurality of electrodes 516 and/or plurality of conductors 515 are coupled to structure 530 via plurality of fixation points 518. In some examples, fixation points 518 may include one or more threaded holes which mate with a corresponding threads of plurality of electrodes 516, plurality of conductors 515, or shafts coupled to electrodes 516 or conductors 515. While the example of FIG. 5 shows plurality of fixation points 518 as threaded holes, plurality of fixation points 518 may include any suitable mechanisms for coupling plurality of electrodes 516 and/or plurality of conductors 515 to structure 530, including but not limited to press fit (e.g., interference fit), laser welding, reflow of material, etc. Plurality of electrodes 516 and/or plurality of conductors 515 may be attached while structure 530 is substantially flat (e.g., compact configuration 434 as shown in FIG. 4), or after structure 430 is converted to an expanded configuration (e.g., expanded configuration 436 as shown in FIG. 4).

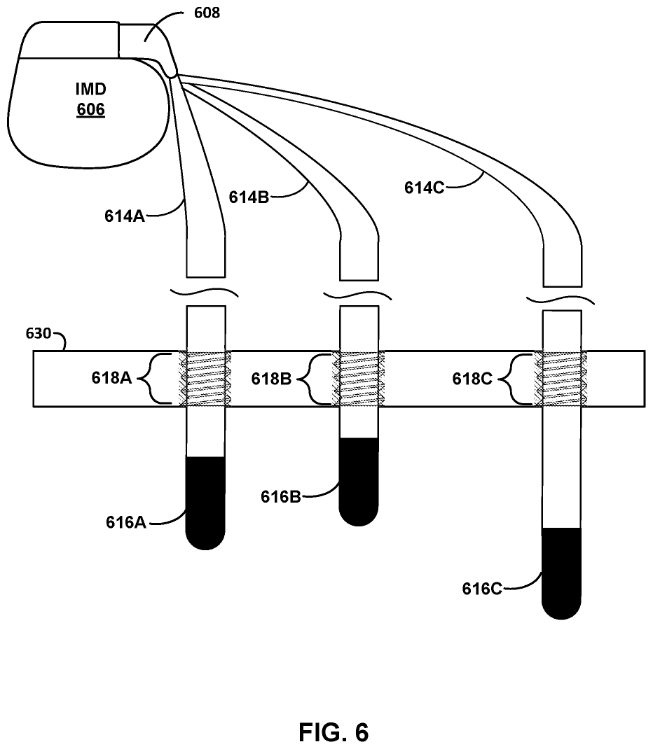

FIG. 6 illustrates an example structure 630, which may be an expandable auxetic structure, including a plurality of fixation points 618 (shown individually as 618A, 618B, and 618C) for coupling a plurality of leads 614 (shown individually as 614A, 614B, and 614C) to structure 630. Structure 630 may be an example of structure 130 or structure 430 shown in connection with FIG. 1 or FIG. 4 respectively. Structure 630 may also be share features with structure 530 as described in FIG. 5, except as described herein. In the example of FIG. 6, an IMD 606 includes plurality of leads 614, which are electrically coupled to IMD 606 via a connector 608, and a plurality of electrodes 616 (shown individually as 616A, 616B, and 616C) disposed on or carried by plurality of leads 614.

In the example of FIG. 6, plurality of leads 614 are coupled to structure 630 via plurality of fixation points 618. However, in some examples, plurality of electrodes 616 are coupled to structure 630 via plurality of fixation points 618 while also still being electrically coupled to plurality of leads 614. In some examples, fixation points 618 may be one or more threaded holes which mate with a corresponding threads of plurality of leads 614. For example, plurality of leads 614 may be screwed or threaded into structure 630 to couple plurality of leads 614 to structure 630. In some examples, plurality of leads 614 may subsequently be separated from structure 630 by unscrewing or unthreading plurality of leads 614 from plurality of fixation points 618. In some examples, plurality of leads 614 can be coupled to structure 630 such that plurality of electrodes 616 extend away from structure 630 by a defined length, which may vary (e.g., such that plurality of electrodes 616 penetrate tissue of a patient at a target depth). While the example of FIG. 6 shows plurality of fixation points 618 as threaded holes, plurality of fixation points 618 may include any suitable mechanisms for coupling plurality of leads 614 or plurality electrodes 618 to structure 630, including but not limited to press fit (e.g., interference fit), laser welding, reflow of material, etc.

Although the example of FIG. 6 shows plurality of leads 614 as being three leads that each carry one electrodes, plurality of leads can include any suitable number of leads (e.g., one, two, three, or more depending on the target region for therapy and the type of therapy being delivered). Similarly, although plurality of electrodes 616 is only shown as three total electrodes, plurality of electrodes 616 can include any number of combination of electrodes. For example, each lead of plurality of leads 614 may carry one or more electrodes (e.g., one, two, three, four, or more electrodes depending on the target region for therapy and the type of therapy being delivered).

In some examples, plurality of leads 614 or plurality of electrodes 616 are attached to structure 630 via attachment at plurality of fixation points 618 when structure 630 is in a compact configuration (compact configuration 434 of FIG. 4). For example, a first lead 614A is attached to structure 630 via first fixation point 618A such that electrode 616A is disposed a first distance away from structure 630 (e.g., a first distance measured from a center of electrode 616A to a surface of structure 630), a second lead 614B is attached to structure 630 via second fixation point 618B such that electrode 616B is disposed a second distance away from structure 630 (e.g., a second distance measured from a center of electrode 616B to a surface of structure 630), and third lead 614C is attached to structure 630 via third fixation point 618C such that electrode 616C is disposed at a third distance away from structure 630 (e.g., a third distance measured from a center of electrode 616C to a surface of structure 630). Plurality of leads 614 may be attached while structure 630 is substantially flat (e.g., compact configuration 434 as shown in FIG. 4), or after structure 630 is converted to an expanded configuration (e.g., expanded configuration 436 as shown in FIG. 4).

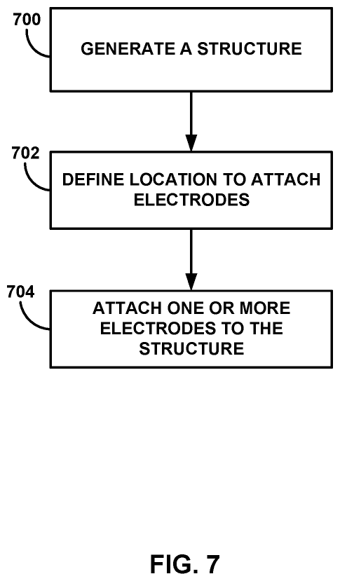

FIG. 7 is a flow chart describing an example technique for making a structure (e.g., any of structures 130, 430, 530, and 630), and is described with respect to structure 430 of FIG. 4. In some examples, a method includes generating a structure 430 (700). As described above, structure 430 may be a deployable auxetic structure. Structure 430 is transformable between a compact configuration 434 and an expanded configuration 436. In some examples, generating structure 430 includes creating plurality of unit cells 438 within structure 430, which may be a plurality of auxetic unit cells. When creating plurality of unit cells 438 within structure 430, structure 430 may be in a first configuration (e.g., compact configuration 434). For example, generating structure 430, including making incisions to create unit cells 438, may occur when structure 430 is in compact configuration 434 and major surface 432 is substantially flat, which may simplify the manufacturing process and space needed. As described above, creating unit cells 438 may include creating incisions in the structure through any suitable technique (e.g., physical cutting, etching, chemical material removal, laser cutting, or the like). In some examples, when incisions are made in structure 430, material may also be removed from structure 430 such that each unit cell of unit cells 438 defines one or more gaps (e.g., voids of material) within each until cell. Incisions into unit cells 438 may define plurality of elements 446.

Continuing with the example of FIG. 7, the technique further includes defining one or more locations to attach plurality of electrodes 116 (702). One or more locations to attach plurality of electrodes 116 (e.g., as shown in FIG. 1) may include plurality of fixation points 418. In some examples, locations for plurality of fixation points 418 may be determined before attaching plurality of electrodes 116 to structure 430. Defining one or more locations to attach plurality of electrodes 116 may include creating a plurality of fixation points 418. As discussed previously, each fixation point of the plurality of fixation points 418 for plurality of electrodes 416 or lead(s) 114 (e.g., as shown in FIG. 1) may correspond to one or more pre-determined target location for placement of one or more electrodes or one or more leads that carry one or more electrodes. When one or more electrodes are disposed on structure 430, the locations of the electrodes on structure 430 may corresponding to one or more pre-determined target locations within a patient for therapy delivery. For example, a generic mapping or a patient-specific mapping of a resected tumor bed (e.g., from a computerized tomography (CT) scan) may be provided, such that unit cells 438 are created to correspond to a three-dimensional contour of structure 430 that maps or otherwise fits the patient-specific mapping. In some examples, locations for placement of electrodes 116 on structure 430 may define a specific electrode array but may not be otherwise specific to a patient's specific tumor void.

Continuing with the example of FIG. 7, the technique further includes attaching one or more electrodes 116 to structure 430 (704). Structure 430 may include a plurality of fixation points 418 for attaching one or more electrodes 116 or one or more leads 114 (e.g., as shown in FIG. 1). Plurality of fixation points 418 may include a threaded hole or another attachment mechanisms such as, but not limited to, press fit (e.g., interference fit), laser welding, reflow of material, etc. In some examples, attaching one or more electrodes 116 to structure 430 includes affixing electrodes 116 to structure 430 via one or more threaded holes, where the threaded holes are located at plurality of fixation points 418.

Additionally or alternatively, one or more electrodes 116 may be defined by structure 430 by removing insulative material to expose conductive material on a surface of structure 430. Attaching one or more electrodes 116 to structure may occur while structure 430 is in compact configuration 434 (e.g., when structure 430 is substantially flat). Additionally or alternatively, attaching electrodes 116 may occur after structure 430 is deployed to expanded configuration 436.

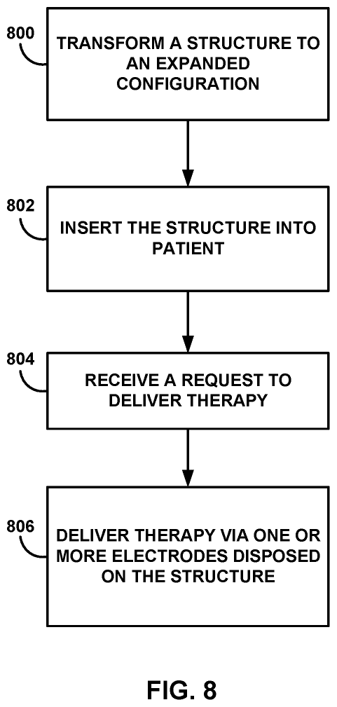

FIG. 8 is a flow chart describing an example technique for implanting and/or inserting a transformable structure into a patient, and is described with respect to system 100 of FIG. 1, IMD 106 of FIG. 1 and FIG. 2, programmer 104 of FIG. 3, and structure 430 of FIG. 4. In the example of FIG. 8, the technique includes transforming (e.g., expanding) a transformable structure 430 from a compact configuration to an expanded configuration (800). Expansion of structure 430 may occur inside patient 112 (e.g., inside cranium 122), or expansion of structure 430 may occur outside of patient 112. For example, expansion of structure 430 may occur before and/or after inserting the structure 430 into patient 112. In some examples, structure 430 is partially deployed and/or expanded before inserting structure 430 into patient 112, and/or further expanded to a fully deployed configuration after inserting structure 430 into patient.

Continuing with the example of FIG. 8, the technique further includes inserting the transformable structure 430 into a patient 112 (802). Structure 430 may be inserted into brain 120 of patient 112. Structure 430 may be implanted within the right and/or the left hemispheres of patient 112 in order deliver therapy to one or more regions of brain 120 and/or sense one or more patient parameters via one or more electrodes 116 disposed on structure 430. In some examples, one or more electrodes 116 are separated from structure 430 and remain in brain 120 of patient 112, and structure 430 is removed. However, in some examples, one or more electrodes remain attached to structure 430 and structure 430 remains implanted in patient 112.

Continuing with the example of FIG. 8, the technique further includes receiving, via processing circuitry (e.g., processing circuitry 210 of IMD 106 or processing circuitry 310 of programmer 104), a request to deliver therapy (e.g., alternating electric field (AEF) or focused tumor treating field (FTTF) therapy) (804). In some examples system 100 includes processing circuitry configured to receive a request to deliver alternating electric field (AEF) or focused tumor treating field (FTTF) therapy. The request may be via user input (such as through programmer 104) and/or an automated system request to start AEF or FTTF therapy delivery.

Continuing with the example of FIG. 8, the technique further includes delivering therapy via one or more electrodes disposed on a transformable structure 130 (806). As described previously, structure 130 is configured to have compact configuration (e.g., compact configuration 434) in which structure 430 is substantially flat. Structure 130 is transformable (or deployable) to an expanded configuration (e.g., expanded configuration 436) in which structure 430 defines one or more three-dimensional contours. In some examples, therapy is delivered via one or more electrodes 116 disposed on structure 130.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

One or more examples are described herein.

Example 1. A system comprising: a structure comprising a pattern of repeating unit cells configured to convert the structure between a compact configuration and an expanded configuration, wherein: the structure is substantially flat in the compact configuration, the structure defines one or more three-dimensional contours in the expanded configuration, and each unit cell of the repeating unit cells comprises a plurality of elements having increased distances between adjacent elements of the plurality of elements in the expanded configuration as compared to the compact configuration; and one or more electrodes disposed on the structure.

Example 2. The system of example 1, wherein the repeating unit cells comprises a plurality of auxetic unit cells.

Example 3. The system of example 2, wherein one or more auxetic unit cells of the plurality of auxetic unit cells is configured to expand in response to a force.

Example 4. The system of examples 1 to 3, wherein the one or more electrodes are configured to deliver focused tumor treating field (FTTF) therapy.

Example 5. The system of examples 1 to 4, wherein when the structure is transformed to the expanded configuration, the one or more electrodes are configured to deliver therapy to one or more pre-determined target locations within a patient.

Example 6. The system of examples 1 to 5, wherein the one or more electrodes are defined by respective portions of the structure.

Example 7. The system of examples 1 to 6, wherein the one or more electrodes are coupled to the structure.

Example 8. The system of examples 1 to 7, wherein the one or more electrodes are coupled to respective different positions on the structure corresponding to one or more pre-determined target locations within a patient for therapy delivery when the structure is in the expanded configuration.

Example 9. The system of examples 1 to 8, wherein the structure is a bi-stable structure comprising a first stable configuration and a second stable configuration, wherein the bi-stable structure is unstable in configurations between the first stable configuration and the second stable configuration, wherein the compact configuration is the first stable configuration, and wherein the expanded configuration is the second stable configuration.

Example 10. A method comprising: generating a structure, the structure comprising a pattern of repeating unit cells configured to convert the structure between a compact configuration and an expanded configuration, wherein: the structure is substantially flat in the compact configuration, the structure defines one or more three-dimensional contours in the expanded configuration, and each unit cell of the repeating unit cells comprises a plurality of elements having increased distances between adjacent elements of the plurality of elements in the expanded configuration as compared to the compact configuration; and attaching one or more electrodes to the structure.

Example 11. The method of example 10, wherein the repeating unit cells comprises a plurality of auxetic unit cells.

Example 12. The method of examples 10 to 11, wherein when generating the structure, the structure is in the compact configuration.

Example 13. The method of examples 10 to 12, wherein generating the structure comprises: creating incisions in the structure to define the pattern of repeating unit cells.

Example 14. The method of examples 11 to 13, wherein one or more auxetic unit cells of the plurality of auxetic unit cells is configured to expand in response to a force.

Example 15. The method of examples 10 to 14, wherein one or more three-dimensional contours comprise one or more pre-determined contours generated from anatomy of a patient.

Example 16. The method of examples 10 to 15, wherein attaching the one or more electrodes to the structure comprises at least: affixing the one or more electrodes to the structure via one or more threaded holes.

Example 17. The method of examples 10 to 16, wherein when the structure is transformed to the expanded configuration, the one or more electrodes are configured to deliver therapy to one or more pre-determined target locations within a patient.

Example 18. The method of examples 10 to 17, wherein the structure is a bi-stable structure comprising a first stable configuration and a second stable configuration, wherein the bi-stable structure is unstable in configurations between the first stable configuration and the second stable configuration, wherein the compact configuration is the first stable configuration, and wherein the expanded configuration is the second stable configuration.

Example 19. A system comprising: a bi-stable auxetic structure comprising a pattern of repeating auxetic unit cells configured to convert the bi-stable auxetic structure between a compact configuration and an expanded configuration upon application of a tension force to the bi-stable auxetic structure, wherein: the bi-stable auxetic structure is substantially flat in the compact configuration, the bi-stable auxetic structure defines one or more three-dimensional contours in the expanded configuration, each auxetic unit cell of the pattern of repeating auxetic unit cells comprises a plurality of elements having increased distances between adjacent elements of the plurality of elements in the expanded configuration as compared to the compact configuration, the compact configuration is a first stable configuration, the expanded configuration is a second stable configuration, and the bi-stable structure is unstable in configurations between the first stable configuration and the second stable configuration; and one or more electrodes disposed on the structure in one or more locations corresponding to pre-determined target locations within a patient for therapy delivery.

Example 20. The system of example 19, wherein one or more three-dimensional contours are one or more pre-determined contours generated from anatomy of a patient.

Example 21. A method comprising: transforming a transformable structure to an expanded configuration; inserting the transformable structure into a patient; receiving, via processing circuitry, a request to deliver therapy; and delivering therapy via one or more electrodes disposed on the transformable structure, wherein: the structure comprises a pattern of repeating unit cells configured to convert the structure between a compact configuration and an expanded configuration, the structure is substantially flat in the compact configuration, the structure defines one or more three-dimensional contours in the expanded configuration, and each unit cell of the repeating unit cells comprises a plurality of elements having increased distances between adjacent elements of the plurality of elements in the expanded configuration as compared to the compact configuration; and one or more electrodes disposed on the structure.

Example 22. The method of example 21, wherein the repeating unit cells comprises a plurality of auxetic unit cells.

Example 23. The method of example 22, wherein one or more auxetic unit cells of the plurality of auxetic unit cells is configured to expand in response to a force.

Example 24. The method of any of examples 21 to 23, wherein the one or more electrodes are configured to deliver Focused Tumor Treating Fields (FTTF) therapy.

Example 25. The method of any of examples 21 to 24, wherein when the structure is transformed to the expanded configuration, the one or more electrodes are configured to deliver therapy to one or more pre-determined target locations within a patient.

Example 26. The method of any of examples 21 to 25, wherein the electrodes are defined by respective portions of the structure.

Example 27. The method of any of examples 21 to 26, wherein the electrodes are disposed on the structure.

Example 28. The method of any of examples 21 to 27, wherein the one or more electrodes are coupled to respective different positions on the structure corresponding to one or more pre-determined target locations within a patient for therapy delivery when the structure is in the expanded configuration.

Example 29. The method of any of examples 21 to 28, wherein the structure is a bi-stable structure comprising a first stable configuration and a second stable configuration, wherein the bi-stable structure is unstable in configurations between the first stable configuration and the second stable configuration, wherein the compact configuration is the first stable configuration, and wherein the expanded configuration is the second stable configuration.

Example 30. A system comprising means for performing the method of any of examples 21-29.

1. A system comprising:
a structure comprising a pattern of repeating unit cells configured to convert the structure between a compact configuration and an expanded configuration, wherein:
the structure is substantially flat in the compact configuration, the structure defines one or more three-dimensional contours in the expanded configuration, and
each unit cell of the repeating unit cells comprises a plurality of elements having increased distances between adjacent elements of the plurality of elements in the expanded configuration as compared to the compact configuration; and
one or more electrodes disposed on the structure.

2. The system of claim 1, wherein the repeating unit cells comprises a plurality of auxetic unit cells.

3. The system of claim 2, wherein one or more auxetic unit cells of the plurality of auxetic unit cells is configured to expand in response to a force.

4. The system of claim 1, wherein when the structure is transformed to the expanded configuration, the one or more electrodes are configured to deliver therapy to one or more pre-determined target locations within a patient.

5. The system of claim 1, wherein the structure is a bi-stable structure comprising a first stable configuration and a second stable configuration, wherein the bi-stable structure is unstable in configurations between the first stable configuration and the second stable configuration, wherein the compact configuration is the first stable configuration, and wherein the expanded configuration is the second stable configuration.

6-8. (canceled)

9. The system of any of claim 1, wherein the one or more electrodes are defined by respective portions of the structure.

10. (canceled)

11. The system of claim 1, wherein the one or more electrodes are coupled to the structure.

12. The system of claim 1, wherein the one or more electrodes are coupled to respective different positions on the structure corresponding to one or more pre-determined target locations within a patient for therapy delivery when the structure is in the expanded configuration.

13. (canceled)

14. The system of claim 1, wherein the one or more electrodes are configured to delivery therapy that includes focused tumor treating field (FTTF) therapy.

15. (canceled)

16. A method comprising:
generating a structure, the structure comprising a pattern of repeating unit cells configured to convert the structure between a compact configuration and an expanded configuration, wherein:
the structure is substantially flat in the compact configuration,
the structure defines one or more three-dimensional contours in the expanded configuration, and
each unit cell of the repeating unit cells comprises a plurality of elements having increased distances between adjacent elements of the plurality of elements in the expanded configuration as compared to the compact configuration; and attaching one or more electrodes to the structure.

17. The method of claim 16, wherein the repeating unit cells comprises a plurality of auxetic unit cells.

18. The method of claim 17, wherein one or more auxetic unit cells of the plurality of auxetic unit cells is configured to expand in response to a force.

19. The method of claim 16, wherein when generating the structure, the structure is in the compact configuration.

20. The method of claim 16, wherein generating the structure comprises:
creating incisions in the structure to define the pattern of repeating unit cells.

21. The method of claim 16, wherein one or more three-dimensional contours comprise one or more pre-determined contours generated from anatomy of a patient.

22. The method of claim 16, wherein attaching the one or more electrodes to the structure comprises at least affixing the one or more electrodes to the structure via one or more threaded holes.

23. The method of claim 16, wherein when the structure is transformed to the expanded configuration, the one or more electrodes are configured to deliver therapy to one or more pre-determined target locations within a patient.

24. The method of claim 16, wherein the structure is a bi-stable structure comprising a first stable configuration and a second stable configuration, wherein the bi-stable structure is unstable in configurations between the first stable configuration and the second stable configuration, wherein the compact configuration is the first stable configuration, and wherein the expanded configuration is the second stable configuration.

25. A system comprising:
a bi-stable auxetic structure comprising a pattern of repeating auxetic unit cells configured to convert the bi-stable auxetic structure between a compact configuration and an expanded configuration upon application of a tension force to the bi-stable auxetic structure, wherein:
the bi-stable auxetic structure is substantially flat in the compact configuration,
the bi-stable auxetic structure defines one or more three-dimensional contours in the expanded configuration,
each auxetic unit cell of the pattern of repeating auxetic unit cells comprises a plurality of elements having increased distances between adjacent elements of the plurality of elements in the expanded configuration as compared to the compact configuration,
the compact configuration is a first stable configuration,
the expanded configuration is a second stable configuration, and
the bi-stable structure is unstable in configurations between the first stable configuration and the second stable configuration; and
one or more electrodes disposed on the structure in one or more locations corresponding to pre-determined target locations within a patient for therapy delivery.

26. The system of claim 25, wherein one or more three-dimensional contours are one or more pre-determined contours generated from anatomy of a patient.
